(19) United States
(12) Patent Application Publication
(10) Pub. No.: US 2021/0077366 A1
(43) Pub. Date: Mar. 18, 2021

(54) HIGH SPF SUNSCREEN COMPOSITIONS

(71) Applicant: Vi-Jon, LLC, St. Louis, MO (US)

(72) Inventors: Sophie Bonham, Maplewood, MO (US); Denisha Patel, Murfreesboro, TN (US)

(21) Appl. No.: 16/794,311

(22) Filed: Feb. 19, 2020

Related U.S. Application Data
(60) Provisional application No. 62/900,041, filed on Sep. 13, 2019.

(51) Int. Cl. A61K 8/06; A61K 8/25; A61K 8/27; A61K 8/29; A61K 8/34; A61K 8/36; A61K 8/37; A61K 8/44; A61K 8/49; A61Q 19/08; A61Q 17/04

(52) U.S. Cl. CPC A61K 8/062; A61K 2800/59; A61K 8/27; A61K 8/29; A61K 8/345; A61K 8/347; A61K 8/361; A61K 8/375; A61K 8/445; A61K 8/4966; A61Q 19/08; A61Q 17/04; A61K 2800/30; A61K 2800/412; A61K 8/25

(57) ABSTRACT

Improved high SPF sunscreen formulations not containing oxybenzone and octinoxate are disclosed. The present disclosure also discloses sunscreen compositions including: (i) a film forming component including at least one film forming polymer; (ii) silica; (iii) a sunscreen active component including at least one sunscreen active compound selected from inorganic sunscreen actives, organic sunscreen actives, and combinations thereof; (iv) a carrier; (v) an optional emulsifier component including at least one emulsifier; (vi) an optional emulsion stabilizer component including at least one emulsion stabilizer; (vii) an optional chelator component; and (viii) an optional skin conditioning agent component.

### CROSS-REFERENCE TO RELATED APPLICATION

This application claims priority to U.S. Provisional Application No. 62/900,041 filed Sep. 13, 2019, the contents of which are incorporated in their entirety.

### FIELD

The field of the disclosure relates generally to sunscreen compositions.

### BACKGROUND

Sunscreen compositions are useful to protect against skin cancer and to inhibit premature aging. Certain sunscreen active components may be problematic. For instance, oxybenzone and octinoxate have been implicated in coral bleaching and water contamination. A need exists for improved sunscreen formulations that provide high SPF while reducing unwanted effects.

This section is intended to introduce the reader to various aspects of art that may be related to various aspects of the disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

### SUMMARY

One aspect of the present disclosure is directed to sunscreen compositions comprising: (i) a film forming component comprising at least one film forming polymer; (ii) silica; (iii) a sunscreen active component comprising at least one sunscreen active compound selected from inorganic sunscreen actives, organic sunscreen actives, and combinations thereof; (iv) a carrier; (v) an optional emulsifier component comprising at least one emulsifier; (vi) an optional emulsion stabilizer component comprising at least one emulsion stabilizer; (vii) an optional chelator component; and (viii) an optional skin conditioning agent component. The total film forming component concentration is from about 0.1 wt. % to about 20 wt. %, the weight ratio of total film forming polymer to silica is from about 1.01:1 to about 10:1, the average particle size of the silica is from about 1 μm to about 40 μm, the total sunscreen active component concentration is from about 1 wt. % to about 50 wt. %, the balance of the composition comprises the carrier, and the SPF rating of the sunscreen composition is from 50 to 110.

The sunscreen compositions of the present disclosure may optionally contain no active amount of oxybenzone and octinoxate.

Various refinements exist of the features noted in relation to the above-mentioned aspects of the present disclosure. Further features may also be incorporated in the above-mentioned aspects of the present disclosure as well. These refinements and additional features may exist individually or in any combination. For instance, various features discussed below in relation to any of the illustrated embodiments of the present disclosure may be incorporated into any of the above-described aspects of the present disclosure, alone or in any combination.

### DETAILED DESCRIPTION

The present disclosure relates to sunscreen formulations providing for high SPF in the range of 50 to 110. The sunscreen formulations comprise: (i) a film forming component comprising at least one film forming polymer; (ii) silica; (iii) a sunscreen active component comprising at least one sunscreen active compound selected from inorganic and organic sunscreen actives; and (iv) water.

Film Former

The compositions of the present disclosure comprise a film forming component comprising at least one film forming polymer.

Non-limiting examples of suitable film forming polymers include the following including derivatives thereof and copolymers thereof: polyvinylpyrrolidones (“PVP”); vinyl acetates (“VA”); vinyls (“VP”); polyvinylcaprolactams; poly(vinyl alcohol); polypropylene glycols (“PPG”); polyethylene glycols (“PEG”); polyglycol esters; cellulose acetates; cellulose (e.g., hydroxymethylcellulose, hydroxyethylcellulose, hydroxypropylcellulose); polyurethanes; dimethicones; acrylates; acrylic esters; acrylamides; and polyethylene glycol ethers of fatty alcohols. Non-limiting examples of copolymers include acrylate copolymer, polyacrylate-2 crosspolymer, styrene/acrylic copolymer, acrylates/hydroxyesters acrylates copolymer, styrene/acrylates copolymer, styrene/acrylates/ammonium methacrylate copolymer, acrylates/beheneth-25 methacrylate crosspolymer copolymer, acrylates/steareth-20-25 methacrylate crosspolymer copolymer, acrylic copolymer, polyacrylate-15, VP/hexadecane copolymer, acrylates/C₁₂₋₂₂ alkyl methacrylate copolymer, silicone acrylate copolymer (e.g., acrylate/dimethicone copolymer), dimethicone and dimethicone PEG-10/15 crosspolymer, polyester-7 and neopentylglycol diheptanoate, combinations thereof, and derivatives thereof.

In some aspects, the film forming polymer is selected from styrene/acrylates copolymer, VP/hexadecane copolymer, sodium polyacrylate, acrylates/C₁₂₋₂₂ alkyl methacrylate copolymer, silicone acrylate copolymer (e.g., acrylate/dimethicone copolymer), dimethicone and dimethicone PEG-10/15 crosspolymer, polyester-7 and neopentylglycol diheptanoate, combinations thereof, and derivatives thereof.

In some aspects, the film forming component comprises styrene/acrylates copolymer, optionally in combination with one or more additional film forming polymers.

In any of the various aspects of the disclosure, the total film forming component concentration is about 0.1 wt. %, about 0.5 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 12.5 wt. %, about 15 wt. %, about 20 wt. %, and any range constructed therefrom, such as from about 0.1 wt. % to about 20 wt. %, from about 0.5 wt. % to about 20 wt. %, from about 1 wt. % to about 15 wt. %, from about 2 wt. % to about 15 wt. %, or from about 2 wt. % to about 10 wt. %.

In aspects where the film forming component comprises styrene/acrylates copolymer, the concentration thereof is about 0.25 wt. %, about 0.5 wt. %, about 0.75 wt. %, about 1 wt. %, about 1.25 wt. %, about 1.5 wt. %, about 2 wt. %, about 3 wt. %, about 4 wt. %, about 5 wt. %, about 6 wt. %, about 7 wt. %, about 8 wt. %, about 9 wt. %, or about 10 wt. %, and any range constructed therefrom, such as from about 0.25 wt. % to about 10 wt. %, from about 0.5 wt. % to about 8 wt. %, from about 1 wt. % to about 8 wt. %, or from about 0.75 wt. % to about 6 wt. %.

Silica

Silica within the scope of the present disclosure generally has a particle size of about 1 μm, about 2 μm, about 5 μm, about 7.5 μm, about 10 μm, about 12.5 μm, about 15 μm, about 17.5 μm, about 20 μm, about 22 μm, about 25 μm, about 30 μm, about 35 μm, about 40 μm, or less than 20 μm and greater than 1 μm, and any range constructed therefrom, such as from about 1 μm to about 40 μm, from about 1 μm to about 30 μm, from about 1 μm to about 22 μm, from about 1 μm to about 20 μm, from about 2 μm to about 25 μm, from about 2 μm to about 20 μm, from about 2 μm to about 17.5 μm, from about 5 μm to about 25 μm, from about 10 μm to about 25 μm, from about 5 μm to about 17.5 μm, from about 5 μm to about 15 μm, from about 10 μm to about 17.5 μm, or from about 10 μm to about 15 μm.

In any of the various aspects of the disclosure, the silica concentration is about 0.25 wt. %, about 0.5 wt. %, about 1 wt. %, about 2 wt. %, about 3 wt. %, about 4 wt. %, about 5 wt. %, about 6 wt. %, about 7 wt. %, about 8 wt. %, about 9 wt. %, or about 10 wt. %, and any range constructed therefrom, such as from about 0.25 wt. % to about 10 wt. %, from about 1 wt. % to about 10 wt. %, from about 0.25 wt. % to about 5 wt. %, from about 2 wt. % to about 5 wt. %, or from about 0.5 wt. % to about 3 wt. %.

In any of the various aspects, the weight ratio of film forming polymer to silica is, about 1.01:1, about 1.25:1, about 1.5:1, about 1.75:1, about 2:1, about 2.5:1, about 3:1, about 4, about 5:1, about 6:1, about 7:1, about 8:1, about 9:1, or about 10:1, and any range constructed therefrom, such as from about 1.01:1 to about 10:1, from about 1.01:1 to about 5:1, from about 1.5:1 to about 8:1, or from about 1.75:1 to about 7:1.

In aspects where the film forming component comprises styrene/acrylates copolymer, the weight ratio thereof to silica is, about 1.05:1, about 1.25:1, about 1.5:1, about 1.75:1, about 2:1, about 2.5:1, about 3:1, about 4, about 5:1, about 6:1, about 7:1, or about 8:1, and any range constructed therefrom, such as from about 1.05:1 to about 8:1, from about 1.25:1 to about 6:1, or from about 1.5:1 to about 5:1.

Sunscreen Active

The sunscreen active component of the present disclosure is suitably selected from inorganic sunscreen actives, organic sunscreen actives, and combinations thereof.

Organic sunscreen actives within the scope of the disclosure include, without limitation, para-aminobenzoic acid (PABA), PABA esters (glyceryl PABA, amyldimethyl PABA, octyldimethyl PABA, and 2-ethylhexyl dimethyl PABA), butyl PABA, ethyl PABA, ethyl dihydroxypropyl PABA, benzophenones (oxybenzone, dioxybenzone, sulisobenzone, benzophenone, and benzophenone-1 through 12), cinnamates (octyl methoxycinnamate (“octinoxate”), isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate, 2-ethoxyethyl p-methoxycinnamate and ethyl methoxycinnamate), cinnamate esters, salicylates (homomethyl salicylate, benzyl salicylate, octyl salicylate, glycol salicylate, isopropylbenzyl salicylate, etc.), anthranilates, ethyl urocanate, homosalate, octisalate, dibenzoylmethane derivatives (e.g., avobenzone), octocrylene, octyl triazone, digalloy trioleate, glyceryl aminobenzoate, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutyiphenol, bis-ethylhexyloxyphenol methoxyphenyltriazine, 4-methylbenzylidenecamphor, methylisothiazolinone, amiloxate, mexoryl, bisdisulizole disodium, menthyl anthranilate, bisoctrizole, ensulizole, isopentyl 4-methoxycinnamate, and combinations thereof. In some aspects, the organic sunscreen active component does not comprise an effective amount of oxybenzone, octinoxate, or a combination thereof.

In some aspects, the organic sunscreen active component comprises avobenzone, homosalate, octocrylene, octyl salicylate, or combinations thereof. In some aspects, the organic sunscreen active component consists of avobenzone, homosalate, octocrylene, and octyl salicylate. In such aspects, the avobenzone concentration is suitably about 0.5 wt. %, about 1 wt. %, about 2 wt. %, about 3 wt. %, about 4 wt. %, about 5 wt. %, about 6 wt. %, about 7 wt. %, about 8 wt. %, about 9 wt. %, or about 10 wt. %, and any range constructed therefrom, such as from about 0.5 wt. % to about 10 wt. %, from about 1 wt. % to about 7 wt. %, or from about 2 wt. % to about 4 wt. %. In such aspects, the homosalate concentration is suitably about 0.5 wt. %, about 1 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, or about 25 wt. %, and any range constructed therefrom, such as from about 0.5 wt. % to about 25 wt. %, from about 5 wt. % to about 20 wt. %, or from about 9 wt. % to about 15 wt. %. In such aspects, the octocrylene concentration is suitably about 0.5 wt. %, about 1 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 20 wt. %, or about 25 wt. %, and any range constructed therefrom, such as from about 0.5 wt. % to about 25 wt. %, from about 5 wt. % to about 15 wt. %, from about 5 wt. % to about 10 wt. %, or from about 8 wt. % to about 10 wt. %. In such aspects, the octyl salicylate concentration is suitably about 0.5 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, or about 15 wt. %, and any range constructed therefrom, such as from about 0.5 wt. % to about 15 wt. %, from about 1 wt. % to about 10 wt. %, from about 2 wt. % to about 8 wt. %, from about 3 wt. % to about 6 wt. %, or from about 4 wt. % to about 5 wt. %.

Inorganic sunscreen actives within the scope of the disclosure include, without limitation, metal oxides (e.g., titanium dioxide and zinc oxide), kaolin, talc, and combinations thereof.

In any of the various aspects of the disclosure, the total sunscreen active component concentration is suitably about 1 wt. %, about 5 wt. %, about 10 wt. %, about 15 wt. %, about 20 wt. %, about 25 wt. %, about 30 wt. %, about 35 wt. %, about 40 wt. %, about 45 wt. %, or about 50 wt. %, and any range constructed therefrom, such as from about 1 wt. % to about 50 wt. %, from about 10 wt. % to about 50 wt. %, from about 20 wt. % to about 50 wt. %, from about 20 wt. % to about 40 wt. %, or from about 25 wt. % to about 40 wt. %.

Carrier

In any of the various aspects of the disclosure the compositions comprise a carrier. Non-limiting examples of carriers include water, alcohol (e.g., ethanol), oil, and combinations thereof.

The concentration of an alcohol carrier may suitably be about 1 wt. %, about 2.5 wt. %, about 5 wt. %, about 7.5 wt. %, about 10 wt. %, about 12.5 wt. %, about 15 wt. %, about 17.5 wt. %, or about 20 wt. %, and any range constructed therefrom, such as from about 1 wt. % to about 20 wt. %, from about 5 wt. % to about 20 wt. %, or from about 10 wt. % to about 20 wt. %.

In any of the various aspects of the disclosure, the balance of the sunscreen composition may comprise water. For instance, the water concentration may be about 25 wt. %, about 30 wt. %, about 35 wt. %, about 40 wt. %, about 45 wt. %, about 50 wt. %, about 55 wt. %, about 60 wt. %, about 65 wt. %, about 70 wt. %, or about 75 wt. %, and any range constructed therefrom, such as from about 25 wt. % to about 75 wt. %, or from about 40 wt. % to about 65 wt. %.

Emulsifier

In any of the various aspects of the disclosure, the compositions may comprise an emulsifier component comprising at least one emulsifier. Emulsifiers can reduce the interfacial tension between phases and improve the formulation and stability of an emulsion. The emulsifiers can be nonionic, cationic, anionic, and zwitterionic emulsifiers (See, e.g., McCutcheon's (1986)).

Non-limiting examples of emulsifiers include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, trideceth-6, polyethylene glycol 20 sorbitan monolaurate (polysorbate 20), polyethylene glycol 5 soya sterol, steareth-2, steareth-20, steareth-21, ceteareth-20, cetearyl glucoside, cetearyl alcohol, C₁₂₋₁₃ pareth-3, PPG-2 methyl glucose ether distearate, PPG-5-ceteth-20, bis-PEG/PPG-20/20 dimethicone, dimethicone PEG-10/15 crosspolymer, ceteth-10, polysorbate 80, cetyl phosphate, potassium cetyl phosphate, diethanolamine cetyl phosphate, triethanolamine, polysorbate 60, alkoxylated carboxylic acid (e.g., glyceryl stearate), PEG-100 stearate, arachidyl alcohol, arachidyl glucoside, wax (e.g., beeswax), gums (e.g., xanthan gum), and combinations thereof.

In some aspects, the emulsifier is selected from dimethicone PEG-10/15 crosspolymer, potassium cetyl phosphate, triethanolamine, glyceryl stearate, PEG-100 stearate, arachidyl glucoside, beeswax, xanthan gum, and combinations thereof. In some aspects, the emulsifier component comprises an alkoxylated carboxylic acid emulsifier (e.g., glyceryl stearate) optionally in combination at least one additional emulsifier, such as for instance, dimethicone PEG-10/15 crosspolymer, potassium cetyl phosphate, triethanolamine, PEG-100 stearate, arachidyl glucoside, beeswax, xanthan gum, and combinations thereof.

The emulsifier concentration may suitably be about 0.5 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 15 wt. %, or about 20 wt. %, and any range constructed therefrom, such as for instance, from about 0.5 wt. % to about 20 wt. %, from about 1 wt. % to about 15 wt. %, from about 1 wt. % to about 10 wt. %, from about 1.5 wt. % to about 10 wt. %, or from about 2 wt. % to about 6 wt. %.

In aspects of the disclosure where the emulsifier component comprises a alkoxylated carboxylic acid emulsifier, the alkoxylated carboxylic acid emulsifier concentration suitably be about 0.5 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, or about 15 wt. %, and any range constructed therefrom, such as for instance, from about 0.5 wt. % to about 15 wt. %, from about 0.5 wt. % to about 10 wt. %, from about 0.5 wt. % to about 7 or from about 1 wt. % to about 5 wt. %.

In some aspects where the emulsifier comprises glyceryl stearate, the concentration thereof may suitably be about 0.1 wt. %, about 0.5 wt. %, about 1 wt. %, about 2 wt. %, about 3 wt. %, about 4 wt. %, about 5 wt. %, about 6 wt. %, about 7 wt. %, about 8 wt. %, about 9 wt. %, or about 10 wt. %, and any range constructed therefrom, such as for instance, from about 0.1 wt. % to about 10 wt. %, from about 0.5 wt. % to about 5 or from about 0.5 wt. % to about 2 wt. %. In some aspects where the emulsifier comprises PEG-100 stearate, the concentration thereof may suitably be about 0.1 wt. %, about 0.5 wt. %, about 1 wt. %, about 2 wt. %, about 3 wt. %, about 4 wt. %, about 5 wt. %, about 6 wt. %, about 7 wt. %, about 8 wt. %, about 9 wt. %, or about 10 wt. %, and any range constructed therefrom, such as for instance, from about 0.1 wt. % to about 10 wt. %, from about 0.5 wt. % to about 5 or from about 0.5 wt. % to about 2 wt. %.

Emulsifier Stabilizer

In any of the various aspects of the disclosure, the compositions may comprise an emulsifier stabilizer component comprising at least one emulsifier stabilizer. Such stabilizers function to improve and maintain the stability and shelf life of the compositions of the present disclosure.

Non-limiting examples of suitable stabilizers include fatty acid esters (e.g., glyceryl monostearate, PEG 7 glyceryl cocoate, glycerol stearate, and glycol distearate); lecithin and hydrogenated lecithin; surfactants (e.g., sodium cocoyl glycinate); polymers (e.g., crosslinked copolymer of acrylic acid and a hydrophobic C₁₀₋₃₀ alkyl acrylate co-monomer); saccharides and polysaccharides (e.g., alkylpolyglucoside, aluminum starch octenylsuccinate, and acrachidyl glucoside); castor oil derivatives (e.g., 12-hydroxy stearic acid and hydrogenated castor oil); alcohols (e.g., cetyl alcohol, ceto stearyl alcohol, stearyl alcohol, and behenyl alcohol); fatty acids (e.g., lauric acid, myristic acid, palmitic acid, stearic acid, and oleic acid); and combinations thereof. In some aspects, the stabilizer is selected from polymers, saccharides and polysaccharides, alcohols, and combinations thereof.

The emulsifier stabilizer component concentration may suitably be about 0.5 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 15 wt. %, or about 20 wt. %, and any range constructed therefrom, such as for instance, from about 1 wt. % to about 15 wt. %, or from about 1.5 wt. % to about 10 wt. %.

Chelator

In any of the various aspects of the disclosure, the compositions may comprise a chelator component comprising at least one chelator. Chelators function to bind with metal ions or metallic compounds to form a complex thereby inhibiting adherence to a surface (such as skin or hair) or contamination therefrom.

Non-limiting examples of suitable chelators include: disodium EDTA; tetrasodium EDTA; tetrahydroxypropyl ethylenediamine; natural and non-natural amino acids (e.g., glutamic acid and histidine); organic diacids (e.g., malate); and polypeptides (e.g., phytochelatin). In some aspects, the chelator comprises at least one carboxyl moiety.

The chelator component concentration may suitably be about 0.01 wt. %, about 0.025 wt. %, about 0.05 wt. %, about 0.075 wt. %, about 0.1 wt. %, about 0.1 wt. %, about 0.2 wt. %, or about 0.25 wt. %, and any range constructed therefrom, such as for instance, from about 0.01 wt. % to about 1 wt. %, from about 0.01 wt. % to about 0.5 wt. %, or from about 0.025 wt. % to about 0.25 wt. %.

Skin Conditioning Agent Component

In any of the various aspects of the disclosure, the compositions may comprise a skin conditioning agent component comprising at least one humectant, at least one humectant, at least one emollient, or a combination thereof. Suitable skin moisturizing components include, but are not limited to, polyols, siloxanes, polysaccharides, esters, fats and oils, derivatives thereof, and combinations thereof. In some aspects of the disclosure, certain skin moisturizing components may also function as film formers, emulsifiers and/or stabilizers.

Non-limiting examples of polyols that may be used as skin conditioning components include: glycerin; ethoxylated glycerine; propoxylated glycerine; propylene glycol; dipropylene glycol; butylene glycol; hexylene glycol; pentylene glycol; polypropylene glycol; polyethylene glycol and derivatives thereof caprylyl glycol; sorbitol; hydroxypropyl sorbitol; erythritol; threitol; pentaerythritol; xylitol; glucitol; mannitol; hexane triol (e.g., 1,2,6-hexanetriol); glyceryl stearate; and combinations thereof.

Non-limiting examples of siloxanes that may be used as skin conditioning components in this disclosure include, but are not limited to: dimethicone; cyclomethicone; phenyl trimethicone; phenyl dimethicone; cetyl dimethicone; stearyl dimethicone; caprylyl methicone; amodimethicone; C30-45 alkyl dimethicone; C30-45 alkyl methicone; cetearyl methicone; dimethicone copolyol; cyclopentasiloxane; dimethicone crosspolymers; dimethicone/vinyl dimethicone crosspolymers, and copolymers, derivatives and mixtures thereof; C30-45 alkyl cetearyl dimethicone crosspolymer; cetearyl dimethicone crosspolymer, and copolymers, derivatives and mixtures thereof; dimethicone/phenyl vinyl dimethicone crosspolymer; vinyl dimethicone/lauryl dimethicone crosspolymer; trifluoropropyl dimethicone/trifluoropropyl divinyldimethicone crosspolymer; trimethylsiloxysilicate; trisiloxane; and combinations thereof.

Non-limiting examples of esters that may be used as skin condition components include: neopentyl glycol diheptanoate; neopentyl glycol diethylhexanoate; neopentyl glycol dicaprylate/dicaprate; neopentyl glycol diglycidyl ether; neopentyl glycol dicaprate; neopentyl glycol diisostearate; butyloctyl salicylate; ethylhexyl stearate; diethylhexyl 2,6-naphthalate; and combinations thereof.

A non-limiting example of a polysaccharide that may be used as skin conditioning components include Xanthan gum.

Non-limiting examples of fats and oils that may be used as skin conditioning components in this disclosure include petrolatum, beeswax, shea butter, shea butter oil, cocoa butter, jojoba butter, aloe butter, olive butter, coconut oil, jojoba oil, olive oil, sunflower seed oil, and combinations thereof.

In some aspects, the skin condition agent is selected from glycerin, glyceryl stearate, neopentyl glycol diheptanoate, butyloctyl salicylate, ethylhexyl stearate, diethylhexyl 2,6-naphthalate, beeswax, cetyl dimethicone, caprylyl methicone, cyclopentasiloxane, trimethylsiloxysilicate, trisiloxane, and combinations thereof.

The skin conditioning agent component concentration may suitably be about 0.1 wt. %, about 0.5 wt. %, about 1 wt. %, about 2 wt. %, about 3 wt. %, about 4 wt. %, about 5 wt. %, about 6 wt. %, about 7 wt. %, about 8 wt. %, about 9 wt. %, or about 10 wt. %, and any range constructed therefrom, such as for instance, from about 0.1 wt. % to about 10 wt. %, from about 1 wt. % to about 10 wt. %, from about 2 wt. % to about 8 wt. %, or from about 3 wt. % to about 6 wt. %.

Antioxidant

The compositions of the present disclosure may further comprise an antioxidant. Non-limiting examples of antioxidants include butylated hydroxytoluene (“BHT”), tocopherol, and sodium ascorbyl phosphate.

The antioxidant component content is suitably from about 0.01 wt. % to about 1 wt. %, from about 0.01 wt. % to about 0.5 wt. %, or from about 0.025 wt. % to about 0.25 wt. %.

Other Components

The compositions of the present disclosure may further comprise other components such as fragrances, buffers, and pH adjusting agents (i.e., acid or base), and preservative.

Non-limiting examples of suitable preservatives include chlorpenesin, benzyl alcohol, 1,2-hexandiol, phenethyl alcohol, phenoxyethanol, parabens, methylisothiazolinone, hexamidine diisethionate, benzalkonium chloride, imidazolidinyl urea, phenoxyethanol, and combinations thereof. In some aspects, the preservative is chlorpenesin, benzyl alcohol, or a combination thereof.

SPF Rating

In accordance with some aspects of the present disclosure, it has been discovered that sun protection factor (“SPF”) is positively correlated with the ratio of the film forming component to silica. In any of the various aspect of the disclosure, the SPF rating is about 50, about 60, about 70, about 80, about 90, about 100, about 110, about 120, and any range constructed therefrom, such as from about 50 to about 110, or from about 110 to about 120.

SPF can be measured by applying sunscreen to the skin of a volunteer and measuring how long it takes before sunburn occurs when exposed to an artificial sunlight source. A non-limiting example of such a method involves applying 2 mg/cm² of a sunscreen formula to an area of the mid-back of a human volunteer, allowing the sunscreen to dry for 15 minutes, and administering a series of five increasing doses of UV radiation, simulating sunlight, to skin sites treated with the sunscreen. Another series of five increasing UV radiation doses is applied within a skin area without the sunscreen. After 16 to 24 hours, the irradiated skin sites are examined to determine the SPF. The SPF is the lowest dose of UV radiation that caused mild sunburn in the sunscreen-treated area divided by the lowest dose of UV radiation that caused mild sunburn in the area without sunscreen. The label SPF of a sunscreen formula is typically based on the average SPF for 10 volunteers.

SPF can also be measured in vitro using a spectrometer where the actual transmittance of the sunscreen is measured, along with the degradation of the product due to being exposed to sunlight. In this method, the transmittance of the sunscreen is measured over all wavelengths in the UVB-UVA range of sunlight, i.e., 290-400 nm. A non-limiting example of one method involves in vitro measurements on artificial substrates that simulate the skin surface (e.g., polymethylmethacrylate or fused silica substrates) and computerized mathematical models based on the UV radiation absorbance spectra of the active ingredients. In some such methods, measurements may be made using an Optimetrics SPF-290S Analyzer System.

In some other methods, such as used in the examples herein, SPF may be measured by FDA, 21 CFR § 201.327, subpart (i), SPF Test Procedure, Sunscreen Drug Products for Over the Counter Human Use, Final Monograph, Federal register, Vol. 76, No. 117, Jun. 17, 2011.

### EXAMPLES

SPF Measurement Method

SPF for each formulation was measured as set forth by the FDA, 21 CFR § 201.327, subpart (i), SPF Test Procedure, Sunscreen Drug Products for Over-the-Counter Hum an Use, Final Monograph, Federal Register, Vol. 76, No. 117, Jun. 17, 2011.

The light source used for the measurements was a Xenon Arc Solar Simulator lamp having a continuous light spectrum in the UVA and UVB range (290-400 nanometers). The spectral output of the solar simulator was filtered to meet the spectral output requirements for testing sunscreen drug products for over-the-counter human use according to 21 CFR Part 201.327 (i)(1) and according to the International Sun Protection Factor (SPF) Test Method, May 2006.

A panel of test subjects was selected and who were instructed to avoid additional UV exposure, and to avoid taking any photosensitizing medications during the duration of the SPF study.

Day 1

Test subjects were exposed a series of five UV radiation doses expressed as Joules/square meter, increasing in 25% increments. The doses were administered to two unprotected separate locations on the back of each test subject, just below the shoulder blades and above the belt-line, to determine the initial unprotected Minimal Erythemal Dose (“MEDu”). The MEDu was administered in the following five dose series, with X representing the amount of UV energy projected to produce the test subject's MEDu: Dose 1=0.64X; Dose 2=0.80X; Dose 3=1.00X; Dose 4=1.25X; and Dose 5=1.56×.

Day 2

Subjects were evaluated for MEDu within 16 to 24 hours following completion of the MEDu doses applied on Day 1. MEDu is defined as the quantity of erythema-effective energy, or dose, corresponding to the first site that produced the first unambiguous erythema reaction with clearly defined borders. MEDu was determined according to the following grading scale in Table 1 below where a response of “+” is defined as the MEDu.

TABLE 1

| Response | Standard |
|---|---|
| − | No perceptible erythemal response |
| ? | Barely perceptible erythemal response |
| + | Unambiguous erythemal reaction with clearly defined |
| | borders (MEDu) |
| ++ | Moderate erythema with sharp borders |
| +++ | Dark red erythema with sharp borders |

Following MEDu determination, two test areas (10 cm×5 cm), 50 square centimeter rectangles, were drawn in designated adjacent locations the back of each subject (between the beltline and the shoulder blade) using a template and an indelible marker. The test formulas were applied in one of the test areas and a FDA standard sunscreen was applied in the adjacent test area. A density of 2 mg/cm² of the test and standard sunscreens were applied by “spotting” across the test area and gently spreading, using a finger cot (as specified in FDA, 21 CFR § 201.327, subpart (4)(iii)) until a uniform film was applied to the entire test area. The test sunscreen was permitted to dry a minimum of 15 minutes prior to the Static UV exposures on the standard sunscreen and the 80 minute water immersion evaluation of the test formula.

The 80 minute water immersion scheme was as follows. An indoor Jacuzzi containing clean drinking water meeting the standard in 40 CFR part 141 was maintained at 23° C. to 32° C. Water temperature, air temperature, and relative humidity were recorded. As required under 21 CFR § 201.237, subpart (i) (7) (ii), Determination of Water Resistance, sunscreen was applied to each subject as indicated above, followed by a waiting period of at least 15 minutes. Each subject then performed 20 minutes of moderate activity in the water, followed by subsequent 15 minute rest period, where the test sites were not toweled. The water activity and rest period were repeated until a total of 80 minutes of water immersion was achieved. Thereafter, the sunscreen test areas were then air dried without toweling.

In a Protected Minimal Erythemal Dose (“MEDp”) evaluation, light source exposure to the test areas was then done according to 21 CFR 201.327, subpart (5), UV Exposure. A series of 5 UV radiation doses expressed as Joules/square meter were administered as specified in FDA, 21 CFR, § 201.327, subpart (S)(iii), progressively increasing in increments of 20 or 15 percent as determined by the previously measured MEDu and the expected SPF range of the test product. The MED was administered in a five dose series with X representing the expected amount of UV energy required to produce a MEDp. For the FDA standard sunscreen, based on an expected SPF 15 (FDA Standard Static), the following doses were administered: Dose 1=0.69X; Dose 2=0.83X; Dose 3=1.00X; Dose 4=1.20X; and Dose 5=1.44×. For the sunscreens under evaluation (following the 80 minute water immersion test), based on an expected SPF of 110.0, the following doses were administered: Dose 1=0.76X; Dose 2=0.87X; Dose 3=1.00X; Dose 4=1.15X; and Dose 5=1.32×.

The Day 1 MEDu measurement was repeated on Day 2. In particular, a second timed series of 5 UV doses, increasing in 25% increments to an unprotected area of the subject's back to determine the second day MEDu of each subject. The series of 5 doses included the original MEDu from Day 1 were repeated (Dose 1=0.64X; Dose 2=0.80X; Dose 3=1.00X; Dose 4=1.25X; and Dose 5=1.56×).

Day 3

On day 3, the MED for each subject for all sites that received UV doses on day 2 (both protected and unprotected areas) were evaluated. The study was conducted in a double-blinded manner. None of the test subjects, the technician who applied the sunscreen products and administered the doses of radiation, and the different grading technician who evaluated the MED responses knew which sunscreen formulation was applied to which site or what doses of UV radiation were administered. The grader evaluated and recorded the MED responses on both the unprotected and protected test sites under the following conditions: the source of illumination was a warm white fluorescent light bulb that provided a level of illumination of at least 450 lux at the test site; and the test subject was seated when evaluated, the same as when the test sites were irradiated.

Calculation of SPF Values

SPF values were calculated for both the test product and the FDA standard using FDA, 21 CFR § 201.327, subpart (i) (6), Determination of SPF, by calculating the ratio of the MEDp value produced in the sunscreen protected sites to the MEDu produced in the unprotected test area, for each individual using the following calculation:

(MEDp)/(MEDu)=SPF value

Data from the subjects was used for calculating the test product's label SPF value. The mean SPF value (x) and the Standard Deviation (s) for these subjects was computed. Based on the subject test panel, the upper 5-percent point from the student distribution table (denoted by t) with n−1 degrees of freedom was obtained. The quantity A was computed using the formula A=ts/Square root n (with n representing the number of test subjects (10) in a full study). A label SPF value was calculated by determining the largest whole number less than X−A.

### Example 1

Sunscreen composition Formulation 1 reported in Table 2 was prepared. In Table 1, Arlacel™ 165 is glycerol monostearate and silica is Kobo MSS-500W characterized by an average particle size of 12 μm. The SPF was determined to be 55.

TABLE 2

| Component | Function | Amount (wt. %) |
|---|---|---|
| Avobenzone | Active | 3.0 |
| Homosalate | Active | 12.0 |
| Octyl Salicylate | Active | 5.0 |
| Octocrylene | Active | 10.0 |
| Water | Carrier | 49.12 |
| Glycerin | Humectant | 0.5 |
| Xanthan Gum | Emulsifier | 0.15 |
| Disodium EDTA | Chelator | 0.05 |
| Chlorphenesin | Preservative | 0.3 |
| Styrene/Acrylates Copolymer 97% | Film Former | 3.0 |
| Diethylhexyl 2,6-Naphthalene | Emollient | 2.0 |
| Butyloctyl Salicylate | Skin Conditioner | 2.65 |
| Arlacel ™ 165 | Emulsifier | 3.5 |
| Caprylyl Methicone | Skin Conditioner | 0.85 |
| Behenyl Alcohol | Stabilizer | 1.25 |
| Dimethicone and | Skin Conditioner | 0.85 |
| Trimethylsiloxysilicate | | |
| BHT | Antioxidant | 0.08 |
| VP/Hexadecane Copolymer | Film Former | 2.0 |
| Sodium Polyacrylate | Film Former | 0.35 |
| Silica (Kobo MSS-500W) | Particulate | 2.85 |
| Benzyl Alcohol | Preservative | 0.5 |
| Fragrance (Belwood 9318) | Fragrance | 0.03 |

### Example 2

Sunscreen composition Formulation 2 reported in Table 3 was prepared where “CP” refers to copolymer. The SPF was determined to be 70.

TABLE 3

| | | Amount |
|---|---|---|
| Component | Function | (wt. %) |
| Avobenzone | Active | 3.0 |
| Homosalate | Active | 15.0 |
| Octyl Salicylate | Active | 5.0 |
| Octocrylene | Active | 10.0 |
| Water | Carrier | 45.37 |
| Glycerin | Humectant | 1.2 |
| Xanthan Gum | Emulsifier | 0.6 |
| Disodium EDTA | Chelator | 0.05 |
| Chlorphenesin | Preservative | 0.3 |
| Styrene/Acrylates Copolymer 97% | Film Former | 4.8 |
| Diethylhexyl 2,6-Naphthalene | Emollient | 2.0 |
| White Beeswax | Emulsifier | 1.5 |
| Arlacel ™ 165 | Emulsifier | 3.0 |
| Caprylyl Methicone | Skin Conditioner | 1.0 |
| Cetyl Dimethicone | Skin Conditioner | 1.0 |
| Dimethicone and Trimethylsiloxysilicate | Skin Conditioner | 0.75 |
| BHT | Antioxidant | 0.05 |
| Sodium Polyacrylate | Film Former | 0.6 |
| Silica (Kobo MSS-500W) | Particulate | 2.25 |
| Acrylates/C₁₂₋₂₂ Alkyl Methacrylate CP | Film Former | 2.0 |
| Benzyl Alcohol | Preservative | 0.5 |
| Fragrance (Belwood 9318) | Fragrance | 0.03 |

### Example 3

Sunscreen composition Formulation 3 reported in Table 4 was prepared where Pemulen™ TR-1 is a crosslinked copolymer of acrylic acid and a hydrophobic C₁₀₋₃₀ alkyl acrylate co-monomer, and KP-545 is a cyclopentasiloxane and acrylates/dimethicone copolymer available from ShinEtsu. The SPF was determined to be 100.

TABLE 4

| Component | Function | Amount (wt. %) |
|---|---|---|
| Avobenzone | Active | 3.0 |
| Homosalate | Active | 15.0 |
| Octyl Salicylate | Active | 5.0 |
| Octocrylene | Active | 10.0 |
| Water | Carrier | 45.61 |
| Disodium EDTA | Chelator | 0.05 |
| Chlorphenesin | Preservative | 0.25 |
| Styrene/Acrylates Copolymer 97% | Film Former | 5.5 |
| Arlacel ™ 165 | Emulsifier | 3.5 |
| Diethylhexyl 2,6-Naphthalene | Emollient | 6.0 |
| White Beeswax | Emulsifier | 1.2 |
| BHT | Antioxidant | 0.05 |
| Pemulen ™ TR-1 | Stabilizer | 0.16 |
| Acrylates C₁₀₋₃₀ Alkyl Acrylate CP | Stabilizer | 0.15 |
| Triethanolamine | pH Adjuster | 0.25 |
| Silica (Kobo MSS-500W) | Particulate | 2.25 |
| KP-545 | Film Former | 1.5 |
| Benzyl Alcohol | Preservative | 0.5 |
| Fragrance (Belwood 9318) | Fragrance | 0.03 |

### Example 4

Sunscreen composition Formulation 4 reported in Table 5 was prepared. The SPF was determined to be 119.

TABLE 5

| Component | Function | Amount (wt. %) |
|---|---|---|
| Avobenzone | Active | 3.0 |
| Homosalate | Active | 15.0 |
| Octyl Salicylate | Active | 5.0 |
| Octocrylene | Active | 10.0 |
| Water | Carrier | 44.06 |
| Disodium EDTA | Chelator | 0.05 |
| Chlorphenesin | Preservative | 0.25 |
| Styrene/Acrylates Copolymer 97% | Film Former | 5.55 |
| Arlacel ™ 165 | Emulsifier | 3.5 |
| Diethylhexyl 2,6-Naphthalene | Emollient | 6.0 |
| White Beeswax | Emulsifier | 1.2 |
| VP/Hexadecane Copolymer | Film Former | 1.5 |
| BHT | Antioxidant | 0.05 |
| Pemulen ™ TR-1 | Stabilizer | 0.16 |
| Acrylates C₁₀₋₃₀ Alkyl Acrylate CP | Stabilizer | 0.15 |
| Triethanolamine | pH Adjuster | 0.25 |
| Silica (Kobo MSS-500W) | Particulate | 2.25 |
| KP-545 | Film Former | 1.5 |
| Benzyl Alcohol | Preservative | 0.5 |
| Fragrance (Belwood 9318) | Fragrance | 0.03 |

### Example 5

Sunscreen composition Formulation 5 reported in Table 6 was prepared where Chemsil K-12 is Dimethicone PEG-10/15 Crosspolymer. The SPF was determined to be 70.

TABLE 6

| | | Amount |
|---|---|---|
| Component | Function | (wt. %) |
| Avobenzone | Active | 3.0 |
| Homosalate | Active | 15.0 |
| Octyl Salicylate | Active | 5.0 |
| Octocrylene | Active | 10.0 |
| Water | Carrier | 52.25 |
| Glycerin | Humectant | 0.5 |
| Xanthan Gum | Emulsifier | 0.25 |
| Disodium EDTA | Chelator | 0.05 |
| Chlorphenesin | Preservative | 0.3 |
| Potassium Cetyl Phosphate | Emulsifier | 1.0 |
| Styrene/Acrylates Copolymer 97% | Film Former | 2.0 |
| Arlacel ™ 165 | Emulsifier | 1.5 |
| Diethylhexyl 2,6-Naphthalene | Emollient | 2.0 |
| White Beeswax | Emulsifier | 0.75 |
| Ethylhexyl Stearate | Emollient | 0.5 |
| Behenyl Alcohol | Stabilizer | 0.8 |
| Dimethicone and Trisiloxane | Skin Conditioner | 0.75 |
| Cetyl Dimethicone | Skin Conditioner | 0.5 |
| Sodium Polyacrylate | Film Former | 0.25 |
| Chemsil K-12 | Film Former | 0.5 |
| Acrylates/C₁₂₋₂₂ Alkyl Methacrylate CP | Film Former | 0.6 |
| Silica (Kobo MSS-500W) | Particulate | 0.5 |
| Benzyl Alcohol | Preservative | 0.5 |
| Trideceth-6 | Emulsifier | 0.5 |
| Fragrance (CE-171486) | Fragrance | 1.0 |

### Example 6

Sunscreen composition Formulation 6 reported in Table 7 was prepared where Montanov™ 202 is arachidyl alcohol, behenyl alcohol and arachidyl glucoside, and LexFil® Sun is polyester-7 and neopentyl glycol diheptanoate. The SPF was determined to be 50.

TABLE 7

| Component | Function | Amount (wt. %) |
|---|---|---|
| Avobenzone | Active | 3.0 |
| Homosalate | Active | 10.0 |
| Octyl Salicylate | Active | 4.5 |
| Octocrylene | Active | 8.0 |
| Water | Carrier | 61.67 |
| Disodium EDTA | Chelator | 0.05 |
| Chlorphenesin | Preservative | 0.3 |
| Arlacel ™ 165 | Emulsifier | 1.0 |
| Styrene/Acrylates Copolymer 97% | Film Former | 1.3 |
| Montanov ™ 202 | Stabilizer | 1.0 |
| White Beeswax | Emulsifier | 0.75 |
| LexFilm^{RTM} Sun | Film Former | 1.6 |
| Acrylates C₁₀₋₃₀ Alkyl Acrylate CP | Stabilizer | 0.13 |
| Pemulen ™ TR-1 | Stabilizer | 0.15 |
| Potassium Hydroxide | pH Adjuster | 0.3 |
| Silica (Kobo MSS-500W) | Particulate | 0.70 |
| Glycerin | Humectant | 2.0 |
| Aluminum Starch Octenylsuccinate | Stabilizer | 2.0 |
| Benzyl Alcohol | Preservative | 0.5 |
| Tocopherol | Antioxidant | 0.05 |
| Fragrance (Belwood 9318) | Fragrance | 1 |

### Example 7

Sunscreen composition Formulation 7 reported in Table 8 was prepared. The SPF was determined to be 50.

TABLE 8

| Component | Function | Amount (wt. %) |
|---|---|---|
| Avobenzone | Active | 3.0 |
| Homosalate | Active | 10.0 |
| Octyl Salicylate | Active | 4.5 |
| Octocrylene | Active | 8.0 |
| Water | Carrier | 60.07 |
| Disodium EDTA | Chelator | 0.05 |
| Chlorphenesin | Preservative | 0.3 |
| Arlacel ™ 165 | Emulsifier | 2.0 |
| Styrene/Acrylates Copolymer 97% | Film Former | 1.3 |
| Montanov 202 | Stabilizer | 1.0 |
| White Beeswax | Emulsifier | 1.0 |
| Behenyl Alcohol | Stabilizer | 0.5 |
| LexFilm^{RTM} Sun | Film Former | 1.7 |
| Acrylates C₁₀₋₃₀ Alkyl Acrylate CP | Stabilizer | 0.11 |
| Pemulen ™ TR-1 | Stabilizer | 0.14 |
| Potassium Hydroxide | pH Adjuster | 0.08 |
| Silica (Kobo MSS-500W) | Particulate | 1.0 |
| Glycerin | Humectant | 1.25 |
| Aluminum Starch Octenylsuccinate | Stabilizer | 3.3 |
| Benzyl Alcohol | Preservative | 0.5 |
| Tocopherol | Antioxidant | 0.05 |
| Fragrance (Belwood 9318) | Fragrance | 0.15 |

### Example 8

Sunscreen composition Formulation 8 reported in Table 9 was prepared. The SPF was determined to be 50.

TABLE 9

| Component | Function | Amount (wt. %) |
|---|---|---|
| Avobenzone | Active | 3.0 |
| Homosalate | Active | 10.0 |
| Octyl Salicylate | Active | 4.5 |
| Octocrylene | Active | 8.0 |
| Water | Carrier | 62.95 |
| Disodium EDTA | Chelator | 0.05 |
| Chlorphenesin | Preservative | 0.3 |
| Arlacel ™165 | Emulsifier | 1.0 |
| Styrene/Acrylates Copolymer 97% | Film Former | 1.3 |
| Montanov ™ 202 | Stabilizer | 1.0 |
| White Beeswax | Emulsifier | 0.75 |
| LexFilm^{RTM} Sun | Film Former | 1.6 |
| Acrylates C₁₀₋₃₀ Alkyl Acrylate CP | Stabilizer | 0.10 |
| Pemulen ™ TR-1 | Stabilizer | 0.12 |
| Potassium Hydroxide | pH Adjuster | 0.08 |
| Silica (Kobo MSS-500W) | Particulate | 0.7 |
| Glycerin | Humectant | 2.0 |
| Aluminum Starch Octenylsuccinate | Stabilizer | 2.0 |
| Benzyl Alcohol | Preservative | 0.5 |
| Tocopherol | Antioxidant | 0.05 |

### Example 9

Sunscreen composition Formulation 9 reported in Table 10 was prepared. The SPF was determined to be 50.

TABLE 10

| Component | Function | Amount (wt. %) |
|---|---|---|
| Avobenzone | Active | 3.0 |
| Homosalate | Active | 10.0 |
| Octyl Salicylate | Active | 4.5 |
| Octocrylene | Active | 8.0 |
| Water | Carrier | 62.55 |
| Disodium EDTA | Chelator | 0.05 |
| Chlorphenesin | Preservative | 0.3 |
| Arlacel ™ 165 | Emulsifier | 1.0 |
| Styrene/Acrylates Copolymer 97% | Film Former | 1.3 |
| Montanov ™ 202 | Stabilizer | 1.0 |
| White Beeswax | Emulsifier | 0.75 |
| LexFilm^{RTM} Sun | Film Former | 1.6 |
| Acrylates C₁₀₋₃₀ Alkyl Acrylate CP | Stabilizer | 0.10 |
| Pemulen ™ TR-1 | Stabilizer | 0.12 |
| Potassium Hydroxide | pH Adjuster | 0.08 |
| Silica (Kobo MSS-500W) | Particulate | 0.7 |
| Glycerin | Humectant | 2.0 |
| Aluminum Starch Octenylsuccinate | Stabilizer | 2.0 |
| Benzyl Alcohol | Preservative | 0.5 |
| Tocopherol | Antioxidant | 0.05 |
| Fragrance (FFS 135US1) | Fragrance | 0.4 |

### Example 10

Sunscreen composition Formulation 10 reported in Table 11 was prepared. The SPF was determined to be 50.

TABLE 11

| | | Amount |
|---|---|---|
| Component | Function | (wt. %) |
| Avobenzone | Active | 3.0 |
| Homosalate | Active | 9.0 |
| Octyl Salicylate | Active | 4.5 |
| Octocrylene | Active | 9.0 |
| Water | Carrier | 47.65 |
| Glycerin | Humectant | 0.5 |
| Xanthan Gum | Emulsifier | 0.35 |
| Disodium EDTA | Chelator | 0.05 |
| Chlorphenesin | Preservative | 0.3 |
| Potassium Cetyl Phosphate | Emulsifier | 1.0 |
| Styrene/Acrylates Copolymer 97% | Film Former | 0.75 |
| Arlacel ™ 165 | Emulsifier | 1.0 |
| Diethylhexyl 2,6-Naphthalene | Emollient | 2.0 |
| Butyloctyl Salicylate | Skin Conditioner | 1.0 |
| White Beeswax | Emulsifier | 0.75 |
| Ethylhexyl Stearate | Emollient | 0.5 |
| Behenyl Alcohol | Stabilizer | 0.5 |
| Cetyl Dimethicone | Skin Conditioner | 0.5 |
| Sodium Polyacrylate | Film Former | 0.25 |
| Chemsil K-12 | Film Former | 0.5 |
| Acrylates/C₁₂₋₂₂ Alkyl Methacrylate CP | Film Former | 0.5 |
| Silica (Kobo MSS-500W) | Particulate | 0.5 |
| Benzyl Alcohol | Preservative | 0.5 |
| Trideceth-6 | Emulsifier | 0.5 |
| SD Alcohol 40-B | Carrier | 15.0 |

### Example 11

Additional sunscreen compositions were evaluated for SPF. The compositions are summarized in Table 12 below where “Form.” Refers to formulation. Kobo MSS-500/20N silica is characterized by an average particle size of 20 μm.

TABLE 12

| | Form. 11 | Form. 12 | Form. 13 | Form. 14 |
|---|---|---|---|---|
| Component | Wt. % | Wt. % | Wt. % | Wt. % |
| Avobenzone | 3.0 | 3.0 | 3.0 | 3.0 |
| Homosalate | 15.0 | 15.0 | 15.0 | 15.0 |
| Octyl Salicylate | 5.0 | 5.0 | 5.0 | 5.0 |
| Octocrylene | 10.0 | 10.0 | 10.0 | 10.0 |
| Water | 49.61 | 44.28 | 44.06 | 46.31 |
| Disodium EDTA | 0.05 | 0.05 | 0.05 | 0.05 |
| Chlorphenesin | 0.25 | 0.25 | 0.25 | 0.25 |
| Styrene/Acrylates | 0.00 | 5.55 | 5.55 | 5.55 |
| Copolymer 97% | | | | |
| Arlacel ™ 165 | 3.5 | 3.5 | 3.5 | 3.5 |
| Diethylhexyl 2,6- | 6.0 | 6.0 | 6.0 | 6.0 |
| Naphthalene | | | | |
| White Beeswax | 1.2 | 1.2 | 1.2 | 1.2 |
| VP/Hexadecane Copolymer | 1.5 | 6.83 | 1.5 | 1.5 |
| BHT | 0.05 | 0.05 | 0.05 | 0.05 |
| Pemulen ™ TR-1 | 0.16 | 0.16 | 0.16 | 0.16 |
| Acrylates C₁₀₋₃₀ Alkyl | 0.15 | 0.15 | 0.15 | 0.15 |
| Acrylate CP | | | | |
| Triethanolamine | 0.25 | 0.25 | 0.25 | 0.25 |
| Silica (Kobo MSS-500W) | 2.25 | 2.25 | 0.00 | 0.00 |
| Silica (Kobo MSS-500/20N) | 0.00 | 0.00 | 2.25 | 0.00 |
| KP-545 | 1.5 | 1.5 | 1.5 | 1.5 |
| Benzyl Alcohol | 0.5 | 0.5 | 0.5 | 0.5 |
| Fragrance (Belwood 9318) | 0.03 | 0.03 | 0.03 | 0.03 |
| Test subjects | 11 | 8 | 11 | 11 |
| Mean | 113.74 | 108.73 | 115.39 | 113.73 |
| Standard Deviation | 12.32 | 12.84 | 12.84 | 12.31 |
| A = ts/sq root n | 7.14 | 7.44 | 7.44 | 7.13 |
| X − A | 106.6 | 101.28 | 107.95 | 106.59 |
| SPF | 106 | 101 | 107 | 106 |

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

When introducing elements of the present disclosure or the embodiment(s) thereof, the articles “a”, “an”, “the” and “said” are intended to mean that there are one or more of the elements. The terms “comprising,” “including,” “containing” and “having” are intended to be inclusive and mean that there may be additional elements other than the listed elements.

What is claimed is:

1. A sunscreen composition comprising:
(i) a film forming component comprising at least one film forming polymer;
(ii) silica;
(iii) a sunscreen active component comprising at least one sunscreen active compound selected from inorganic sunscreen actives, organic sunscreen actives, and combinations thereof; and
(iv) a carrier,
wherein:
the total film forming component concentration is from about 0.1 wt. % to about 20 wt. %;
the weight ratio of total film forming polymer to silica is from about 1.01:1 to about 10:1;
the average particle size of the silica is from about 1 μm to about 40 μm;
the total sunscreen active component concentration is from about 1 wt. % to about 50 wt. %;
the balance of the composition comprises the carrier; and
the SPF rating of the sunscreen composition is from 50 to 120.

2. The sunscreen composition of claim 1, wherein the film forming polymer concentration is from about 2 wt. % to about 15 wt. %.

3. The sunscreen composition of claim 1, wherein the film forming component comprises styrene/acrylates copolymer, wherein the concentration thereof is from about 1 wt. % to about 8 wt. %.

4. The sunscreen composition of claim 1, wherein the weight ratio of film forming polymer to silica is from about 1.01:1 to about 10:1.

5. The sunscreen composition of claim 3, wherein the weight ratio of styrene/acrylates copolymer to silica is from about 1.05:1 to about 8:1.

6. The sunscreen composition of claim 1, wherein the average particle size of the silica is from about 1 μm to about 30 μm.

7. The sunscreen composition of claim 6, wherein the average particle size of the silica is from about 5 μm to about 17.5 μm.

8. The sunscreen composition of claim 1, wherein the silica concentration is from about 0.25 wt. % to about 10 wt. %.

9. The sunscreen composition of claim 8, wherein the silica concentration is from about 1 wt. % to about 5 wt. %.

10. The sunscreen composition of claim 1, wherein the sunscreen active component does not comprise an effective amount of oxybenzone and octinoxate.

11. The sunscreen composition of claim 1, further comprising:
(v) an emulsifier component comprising at least one emulsifier; and
(vi) an emulsion stabilizer component comprising at least one emulsion stabilizer, wherein:
the emulsifier component concentration is from about 0.5 wt. % to about 20 wt. %; and
the emulsion stabilizer component concentration is from about 0.05 wt. % to about 20 wt. %.

12. The sunscreen composition of claim 11, wherein the emulsifier component concentration is from about 1 wt. % to about 15 wt. %.

13. The sunscreen composition of claim 11, wherein the emulsifier component comprises an alkoxylated carboxylic acid emulsifier, wherein the sunscreen composition comprises from about 0.5 wt. % to about 10 wt. % of the alkoxylated carboxylic acid emulsifier.

14. The sunscreen composition of claim 11, wherein the emulsion stabilizer component concentration is from about 1 wt. % to about 15 wt. %.

15. The sunscreen composition of claim 1, further comprising a chelator component (vii) at a concentration of from about 0.01 wt. % to about 1 wt. %.

16. The sunscreen composition of claim 15, wherein the chelator component comprises a chelator having at least one carboxylic acid moiety.

17. The sunscreen composition of claim 16, wherein the carboxylic acid chelator is an amino acid or EDTA.

18. The sunscreen composition of claim 1, further comprising a skin conditioning agent component (viii) at a concentration of from about 0.1 wt. % to about 10 wt. %.

19. The sunscreen composition of claim 1, wherein the carrier comprises water or wherein the carrier is water.

20. The sunscreen composition of claim 1, wherein the SPF rating is from 110 to 120.
